(19) United States
(12) Patent Application Publication
(10) Pub. No.: US 2024/0132848 A1
(43) Pub. Date: Apr. 25, 2024

(54) METHOD FOR INDUCED DIFFERENTIATION OF EXTENDED PLURIPOTENT STEM CELL INTO CARDIOMYOCYTE AND USE THEREOF

(71) Applicant: HUBEI UNIVERSITY, Wuhan (CN)

(72) Inventors: Donghui Zhang, Wuhan (CN); Li LI, Wuhan (CN); Lin CAI, Wuhan (CN); Zhongjun WAN, Wuhan (CN); Ruxiang WANG, Wuhan (CN)

(73) Assignee: HUBEI UNIVERSITY, Wuhan, HB (CN)

(21) Appl. No.: 18/546,266

(22) PCT Filed: Apr. 28, 2022
(86) PCT No.: PCT/CN2022/090544

§ 371 Date: Aug. 10, 2023

(30) Foreign Application Priority Data
May 16, 2021 (CN) 202110550659.4

(51) Int. Cl. C12N 5/077

(52) U.S. Cl. CPC C12N 5/0657; C12N 2501/115; C12N 2501/15; C12N 2501/415; C12N 2501/727; C12N 2506/45

(57) ABSTRACT

The present disclosure provides a method for induced differentiation of an extended pluripotent stem cell (EPSC) into a cardiomyocyte and use thereof, and belongs to the technical field of biomedicine. A reagent used for the induced differentiation is a culture medium having definite chemical components, which can obtain cardiomyocytes having high purity and stable between-batch differentiation efficiency. Compared with cardiomyocytes differentiated from existing pluripotent stem cells, the cardiomyocyte obtained has strong early proliferation ability, and more cell number can be obtained; after extended culture and construction into engineering heart microtissue, maturity is higher, the structure is more regularly arranged, and functional contractility is enhanced. Therefore, the present disclosure is a suitable for mechanism research of heart diseases, drug screening, and cell therapy, and thus has excellent practical application value.

### CROSS REFERENCE TO RELATED APPLICATION

This patent application is a 371 national stage application of International Patent Application No. PCT/CN2022/090544, filed on Apr. 29, 2022, which claims the benefit and priority of Chinese Patent Application No. 202110550659.4 filed with the China National Intellectual Property Administration on May 17, 2021, and entitled “METHOD FOR INDUCED DIFFERENTIATION OF EXTENDED PLURIPOTENT STEM CELL INTO CARDIOMYOCYTE AND USE THEREOF”, the disclosure of which is incorporated by reference herein in its entirety as part of the present application.

### TECHNICAL FIELD

The present disclosure relates to the technical field of biomedicine, in particular to a new method that could induce differentiation of an extended pluripotent stem cell (EPSC) into a cardiomyocyte and use thereof.

### BACKGROUND

Cardiovascular disease (CVD) is known to have highest global morbidity and mortality. Ischemic heart diseases, rheumatic heart disease, diabetic cardiomyopathy, etc., can lead to cardiomyocyte apoptosis, myocardial remodeling and progressive deterioration, and finally to heart failure. Still no therapeutic drugs for reversing heart failure post-CVD have been clinically approved, and expectation for heart transplantation proves challenging due to the shortage of donors. In recent years, coronary artery bypass and cardiac stent bypass has prolonged the survival of patients to avoid further progression from CVD to heart failure, but the mortality of CVD is rising year by year. The main obstacle to restoring heart physiological function is myocardium regeneration. Unfortunately, cardiomyocyte proliferation is significantly reduced with the aging process (1% per year). Finding effective ways to replenish the cardiomyocytes loss during a cardiac event is quite complex. Existing methods to culture adult cardiomyocytes in vitro hold many challenges, and has not proved an effective way to obtain sufficient cardiomyocytes. Regenerative medicine offers new hope in the field of cardiac disease research, in which pluripotent stem cells derived cardiomyocytes becomes a promising source.

Human induced pluripotent stem cells (hiPSCs) generated by somatic cell reprogramming have the ability to differentiate into different types of cells in the embryo, and overcome the existing challenge of differentiated embryonic stem cells (ESCs) with respect to ethical issues and the risk of immunological rejection. hiPSCs are well-accepted seed cells with transformation potential for regenerative medicine. By regulating the Wnt signaling, induced pluripotent stem cells (iPSCs) can be differentiated into cardiomyocytes, but the directed differentiation efficiency and its immature status limit its application.

In 2017, Yang et al. first established an extended pluripotent stem cell (EPSC) line having totipotent characteristics. The cell line has intraembryonic and extraembryonic tissue developmental potential, exhibits excellent interspecies chimeric ability, and possesses the advantages of single cell passage and multiplication with high proliferation rate (Yang Yang et al. Cell. 2017 6; 169(2): 243-257). In 2020, Ran et al. successfully induced both hESCs and hiPSCs into EPSCs in definite components, using LCDM-LY medium including 6 inhibitors to modify the feeder-free transition and maintenance conditions for human EPSCs. This study further laid an excellent foundation for the transformation application of EPSCs (Ran Zheng et al. bioRxiv.18 Oct. 2020). Considering that Qinming Wang et al. had established a differentiation protocol to efficiently generate functional hepatocytes from EPSCs, EPSCs could be a more promising cell source for generating efficient and stable cardiomyocytes. However, the method for inducing EPSCs into cardiomyocytes with defined components still needs to be developed.

### SUMMARY

In view of the deficiencies in the prior methodologies, the present disclosure aims to provide a method for induced differentiation of an EPSC into a cardiomyocyte and use thereof. In the present disclosure, the EPSC is induced to differentiate into the cardiomyocyte through small molecule-based phased regulation of a Wnt signaling pathway. Therefore, the present disclosure has excellent practical application value.

Specifically, the present disclosure adopts the following technical solutions:

A first aspect of the present disclosure provides use of an EPSC in preparation of a cardiomyocyte.

Specifically, the use includes induced differentiation of the EPSC into the cardiomyocyte.

More specifically, the induced differentiation is implemented by inducing the EPSC to differentiate into the cardiomyocyte through small molecule-based phased regulation of a Wnt signaling pathway.

A second aspect of the present disclosure provides a method for induced differentiation of an EPSC into a cardiomyocyte. The method is implemented by inducing the EPSC to differentiate into the cardiomyocyte through small molecule-based phased regulation of a Wnt signaling pathway.

Specifically, the method includes performing the induced differentiation under a culture medium condition with chemically defined components. Preferably, the culture medium condition with chemically defined components is a replacement of serum with a serum substitute with defined components and growth factors.

Preferably, before the small molecule-based phased regulation, the method further includes rapid transition and transformation of the EPSC via mTeSR™ 1 and/or KnockOut™ DMEM/F-12+B27+fibroblast growth factor 2 (FGF2)+transforming growth factor-β (TGFβ). Further preferably, the B27 cell culture medium additive contains insulin. Culture time is 1-4 days.

Further preferably, if the EPSC reaches at least 50% confluence, preferably greater than 85% confluence, and more preferably at least 95% confluence, the induced differentiation is initiated to form the cardiomyocyte.

Preferably, the small molecule-based phased regulation specifically includes steps of: adding a small molecule CHIR99021 for culturing; and adding Wnt signaling pathway-inhibiting small molecules IWR and IWP2 for culturing.

Herein, a concentration of the CHIR99021 is 5-10 μM, and preferably 7.5 μM; a concentration of the IWR is 2.5-7.5 μM, and preferably 5 μM; and a concentration of the IWP2 is 1-5 μM, and preferably 2.5 μM. Efficiently differentiated cardiomyocytes are obtained by controlling the concentrations and time of added small molecules.

A cell culture medium used during the small molecule-based phased regulation includes 1640+B27. Preferably, the B27 cell culture medium additive is insulin-free.

Preferably, culture time during the small molecule-based phased regulation is 3-7 days, and preferably 4-5 days.

A third aspect of the present disclosure provides a cardiomyocyte obtained by the above culture method. The cardiomyocyte is an EPSC-derived cardiomyocyte, and the cardiomyocyte obtained by the above culture method is large in number and strong in early propagation capability.

A fourth aspect of the present disclosure provides use of the above culture method and/or the above cardiomyocyte in any one or more of the following:
a) preparation of a cardiomyocyte product for relevant basic research;
b) drug screening and cardiac-related disease diagnosis; and
c) cardiac-related disease treatment.

Herein, in the use a), the relevant basic research focuses on structural functions and characteristics of the cardiomyocyte, signaling pathways, and electrophysiological signal detection and research;
in the use b), the drug screening includes drug safety evaluation and research and development of molecules of new drugs; and
in the use c), the cardiac-related disease includes myocardial infarction; and the treatment includes cell therapy.

One or more of the above technical solutions have the following beneficial technical effects:
1. According to the above technical solutions, an EPSC can be successfully differentiated into a high-purity cardiomyocyte for the first time, being detected by using a cardiomyocyte marker cardiac troponin T (cTnT) during the process of differentiation of the EPSC for different days.
2. According to the above technical solutions, comparing cardiomyocytes differentiated from EPSCs with those differentiated from hiPSCs, the cardiomyocytes differentiated from the EPSCs exhibit the fact that more differentiated cardiomyocytes could be obtained under the same bottom area, and in general, the count is greater than 10 million/9.6 cm². After cell re-seeding, the early proliferation ability of the EPSC-differentiated cardiomyocytes was relatively high.
3. With the increase in culture time and after construction into myocardial microtissues, EPSC-differentiated myocardial tissues were more mature and arranged regularly, and also observed polar distribution of cell junction, and enhanced functional contractility.

In conclusion, the above technical solutions will provide more choices for stem cells as seed cells for producing cardiomyocytes, promote the use of differentiation of stem cells into cardiomyocytes in the related field of heart diseases, and have a wide prospect in the aspects of heart disease diagnosis, drug screening, and cell therapy. Therefore, there is excellent practical application value.

### BRIEF DESCRIPTION OF THE DRAWINGS

The accompanying drawings, which constitute a part of the present disclosure, are intended to provide a further understanding of the present disclosure, and the illustrative examples of the present disclosure and the description thereof are intended to explain the present disclosure, and do not constitute an improper limitation on the present disclosure.

FIG. 1 shows a solution of a differentiation process of the present disclosure and light microscopic images of cells cultured for different days.

FIGS. 2A-F show flow cytometry results of different stages of differentiation and immunofluorescence images of different stages in Example 3 of the present disclosure, where A shows an immunofluorescence staining demonstrates the differentiation into mesoblastema on Day 5; B shows an immunofluorescence staining demonstrates cells at mesoblastema stage being myocardial precursor cells on Day 12; C shows an immunofluorescence staining demonstration of the vast majority of differentiated cells being cardiomyocytes on Days 16 to 18; D shows a percentage of mesoblastema detected by the flow cytometry; E shows a percentage of the myocardial precursor cells detected by the flow cytometry; and F shows a percentage of cardiomyocyte marker cTnT+ cells detected by the flow cytometry.

FIGS. 3A-B show related results of cTnT positive rate in Example 3 of the present disclosure, where A illustrates cTnT positive differentiation efficiency without screening in eight different batches; and B is a histogram of multi-batch unscreened average cTnT positive differentiation efficiency, indicating that the cTnT positive rate is all at least 80%.

FIG. 4 shows a statistical and fitting chart of the percentage results of the cardiomyocytes beat area on Day 16 from different cell-initiated confluences under the same differentiation scheme and concentration in Example 4 of the present disclosure.

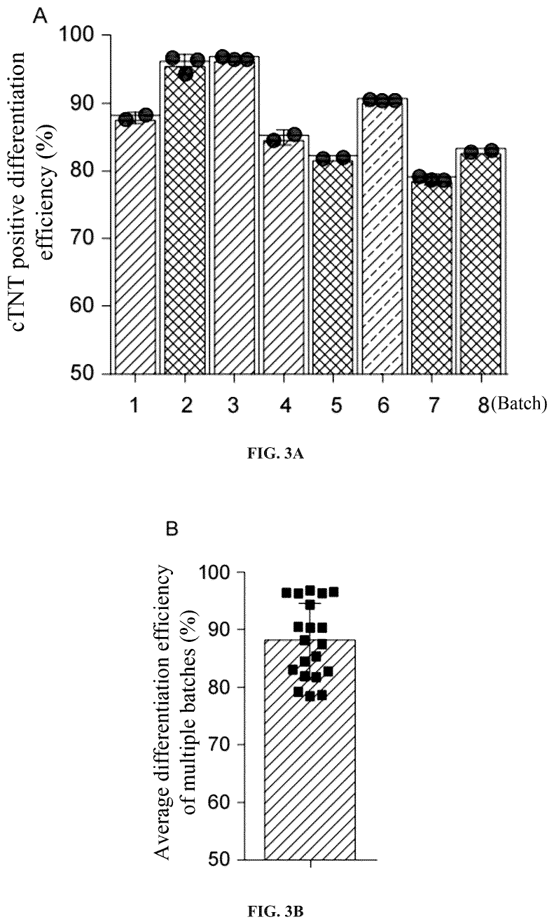

FIG. 5 shows that after extended pluripotent stem cell-derived cardiomyocytes (EPSC-CMs) and human induced pluripotent stem cell-derived cardiomyocytes (hiPSC-CMs) are re-seeded in Example 5 of the present disclosure, the cell proliferation ability of the EPSC-CM at the early stage is slightly high, but there is no significant difference, and the proliferation ability after long-term culture is almost similar, where A illustrates the actual cell staining; and B is a histogram.

FIGS. 6A-B show fluorescence staining results of cardiomyocyte markers α-actinin and cardiac troponin I (cTnI) and maturation markers N-cadherin and ryanodine receptor 2 (RYR2) after cardiac microtissue culture in Example 6 of the present disclosure.

FIGS. 7A-B illustrate a comparison of the functional contractility of cardiac microtissue in EPSCs versus hiPSC-CMs in Example 6 of the present disclosure, where A illustrates a comparison of the maximal contractile amplitude; and B illustrates a comparison of the interval time per contraction.

FIGS. 8A-B illustrate cell residence and survival four weeks after EPSC-CMs are transplanted into the myocardium of a nude rat in Example 7 of the present disclosure, where A is a heart hematoxylin-eosin (H&E) staining result and an enlarged view, and B shows immunofluorescence staining images displaying that resident human cardiomyocytes sarcomere are well arranged and complete.

### DETAILED DESCRIPTION OF THE EMBODIMENTS

It should be noted that the following detailed description is exemplary and is intended to further illustrate the present application. Unless otherwise specified, all technical and scientific terms used herein have the same meaning as commonly understood by one of ordinary skill in the art to which this application belongs.

It should be noted that the terminology used herein is for the purpose of describing particular embodiments only and is not intended to limit exemplary embodiments according to the present disclosure. As used herein, unless otherwise specified herein, the singular forms are also intended to include the plural forms. In addition, it should also be understood that when the terms “comprise” and/or “include” are used in this specification, they specify the presence of features, steps, operations, devices, components, and/or combinations thereof.

As stated above, theoretically, EPSCs should have higher developmental potential than iPSCs, but it is unknown whether EPSCs can be used as seed cells of cardiomyocytes and whether an induction condition with defined components can be used to stably form efficiently differentiated cardiomyocytes that are used in the field of cardiac drug development and myocardial regeneration research. Further exploration is essential.

In view of this, the present disclosure uses a method for differentiation culture with high differentiation efficiency and definite chemical components, namely, EPSCs are induced to differentiate into cardiomyocytes through small molecule-based phased regulation of a Wnt signaling pathway.

According to the findings of the present disclosure, if a set of directly differentiating original hiPSCs into myocardium is used for EPSC differentiation, namely, a differentiation process that uses 1640+B27 mixed with small molecules for regulating the Wnt signaling pathway, cardiomyocytes with the efficiency lower than 10% can be obtained only through differentiation from different cell initiated confluences; moreover, highly-efficient cardiomyocytes cannot be obtained through the initiation period of differentiation by adding activin A and bone morphogenetic protein 4 (BMP4) on the basis of the scheme.

To this end, the present disclosure has the following improvements in the differentiation process of cardiomyocytes: (1) 1640+B27 basal medium and small molecules for regulating the Wnt signaling pathway are changed after the pluripotency of EPSCs is rapidly transited and transformed for 1-4 days by mTeSR™ 1 and the cells reach at least 85% confluence. (2) Two small molecules, IWR and IWP2, are used at the same time while inhibiting Wnt signals in the differentiation process. (3) Further, in view of nearly 100 components in mTeSR™ 1, the effect of rapid transformation and further differentiation can be achieved by using KnockOut™ DMEM/F-12+B27 (+Insulin)+FGF2 (100 ng/mL)+TGFβ (2 μg/mL).

In a specific example of the present disclosure, a cardiomyocyte acquisition method is provided, and the method is performed according to the following process:
1) EPSCs are cultured in an EPSC-specific culture medium, digested with TrypLE, passaged at a ratio of (4-5)×10⁴ cells/cm², and cultured for 1-4 days using the foregoing transition medium.
2) Cells grow to 95% to begin differentiation, the cell culture medium is replaced with 1640+B27 (−Insulin), 7.5 μM small molecule glycogen synthase kinase-3β (GSK3β) inhibitor CHIR99021 is added, and the culture is maintained for two days.
3) CHIR is removed for culture for one day.
4) 5 μM IWR and 2.5 μM IWP2 (small molecules for regulating the Wnt signaling pathway) are added and cultured for two days.
5) IWR and IWP2 are removed from the culture medium.
6) Beating cardiomyocytes can be observed microscopically on Days 10 to 12 of the differentiation of cardiomyocytes.
7) On Day 16, the differentiated cardiomyocytes is digested and re-seeded or microtissues are constructed, and the cTnT positive percentage of differentiated cells is detected by flow cytometry.
8) Cardiomyocyte proliferation is detected. The cells are labeled with 5-ethynyl-2′-deoxyuridne (EdU) for 24 h on Days 3, 7, and 15 after re-seeding, stained immunofluorescent with α-actinin marked cardiomyocytes after 24 h, and the proliferation percentage of cardiomyocytes is counted.
9) Myocardial maturity is determined by immunofluorescence staining and contractile function measurement. Tissues are cultured until Day 14, and immunofluorescently stained with α-actinin, cTnI, N-cadherin, and 4′,6-diamidino-2-phenylindole (DAPI), and the arrangement and structure of two myocardial microtissues are observed. Meanwhile, under electrical stimulation at 1.5 Hz, a video of microtissue contraction is recorded, and the contractility of either group is analyzed by Image J plug-in.

EPSC-CMs obtained by the method of the present disclosure can be used in: (1) structural functions and characteristics of the cardiomyocyte, signaling pathways, and electrophysiological detection and research; (2) drug safety evaluation research and development of molecules of new drugs; and (3) high-efficient acquisition of differentiated mature cardiomyocytes, which can be used in the treatment of related heart diseases like myocardial infarction by cell injection or in combination with tissue engineering.

The present disclosure will be further described with reference to specific examples. The following examples are merely intended to explain the present disclosure, and not to limit the content thereof. If the specific conditions are not specified in the examples, they are usually subject to conventional conditions or the conditions recommended by the sales company; there is no particular limitation in the present disclosure, and everything can be commercially available.

### Example 1

EPSC Culture and Passage

EPSC medium was consisted of a 1:1 mixture of KnockOut™ DMEM/F-12 and Neurobasal™ Medium, which was supplemented with 1% B27(−Vitamin A), 0.5% N₂, 5% serum substitute, 1% glutamate, 1% non-essential amino acid, 1% double antibody, 0.1 mM β-mercaptoethanol, 10 ng/mL recombinant human leukemia inhibitory factor (LIF), 1 μM CHIR99021, 2 μM (S)-(+)-dimethene malate, 2 μM minocycline hydrochloride, 1 μM IWR-endo-1, and 2 μM Y-27632.

EPSCs were cultured by using an EPSC medium and a culture plate coated with Matrigel (3-fold concentration), and passaged with TrypLE™ Express in a single cell clone manner every 3 days. Each digestion lasted for 3-4 min. According to the growth and proliferation states during cell transformation, the cells were passaged at a ratio of 1:2 to 1:8. The cells were blown off into the single cell state as much as possible during each passage. After continuous culture for 10-15 passages, it could be seen that the form of EPSC clone was formed. There were the following features: the clone was stereoscopic, tight, bright, and glossy with a raised spherical shape, the nucleo-cytoplasmic ratio was high, and the expression of some pluripotent genes was significantly upregulated when detected by Q-PCR.

### Example 2

Differentiation of EPSCs into Cardiomyocytes

On Day 0, after EPSCs were digested with TriplE, the cells at a density of (4-5)×10⁴ cells/cm² were provided to resuspend in EPSC medium, and there was no need to be blown off into a single cell state when used for differentiation. A culture plate or Petri dish was coated with Matrigel (normally 1-fold concentration), namely, every 1 μL of Matrigel was diluted and mixed well with 100 μL of pre-cooled DMEM/F-12.

On Day 1, mTeSR™ 1 or KnockOut™ DMEM/F-12 supplemented with 1% B27 (+insulin), 100 ng/mL FGF2, and 2 μg/mL TGFβ was changed. The cell volume began to increase, and the clone extended towards the periphery. The culture medium was changed every day. There was an obvious boundary line between clones until the cells reached at least 95% confluence. The transition culture lasted for about two days.

On Day 3, the culture medium was replaced with 1640+B27 (−insulin), and the cells were induced with small molecule GSK3β inhibitor CHIR99021 (7.5 μM) for two days.

On Day 4, the CHIR99021 was removed and replaced with 1640+B27 (−insulin).

On Days 5 to 6, 5 μM IWR and 2.5 μM IWP2 (small molecules for regulating the Wnt signaling pathway) were added.

On Day 7, small molecules were removed, 1640+B27 (−insulin) were changed, and later, the culture medium was changed every two days.

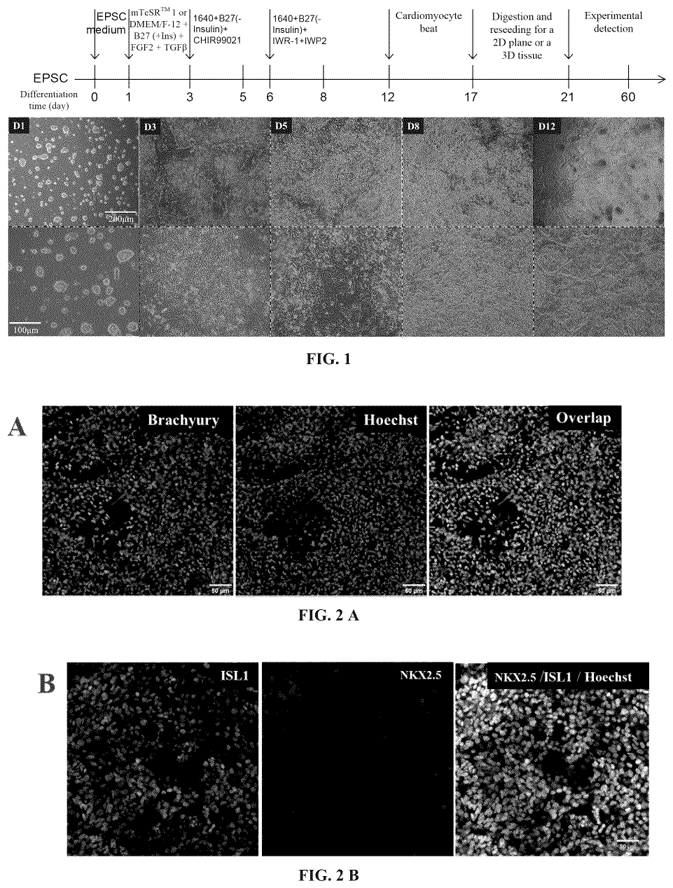

On Days 10 to 12, massive beating cardiomyocytes could be observed microscopically. The specific differentiation flow chart of EPSC and the light microscopic results of the cells differentiated for different time are shown in FIG. 1.

### Example 3

Positive percentages of myocardial precursor cells and cardiomyocytes during the detection of differentiation of EPSCs into cardiomyocytes by flow cytometry and immunofluorescence technique.

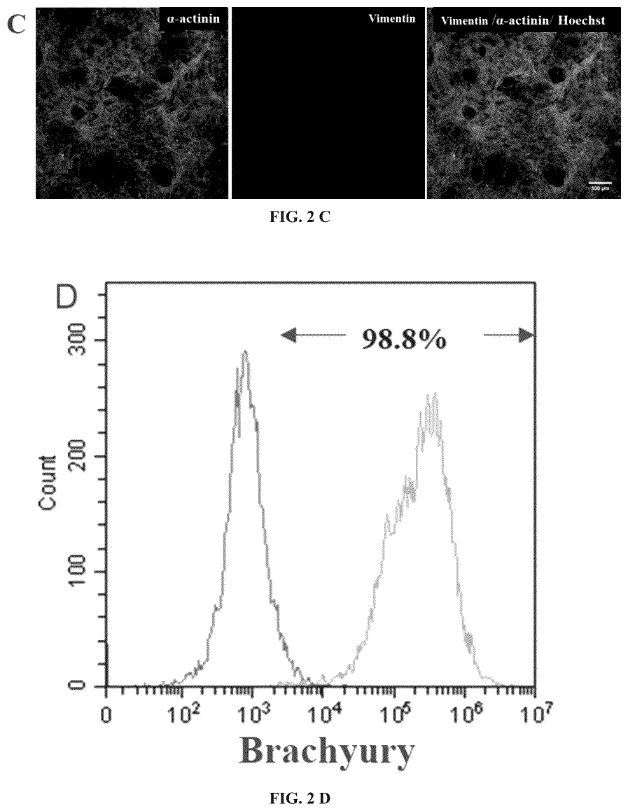

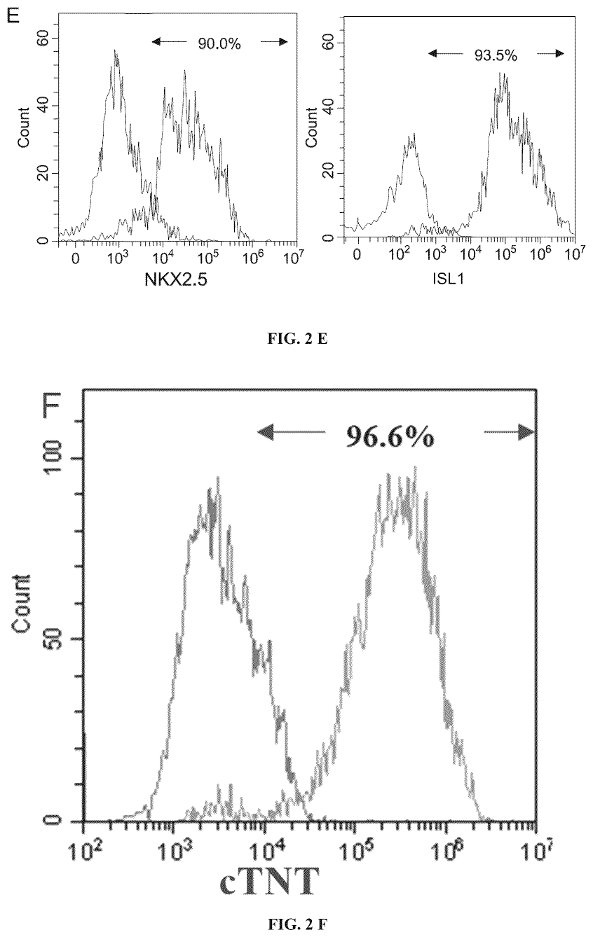

According to the above method, on Day 5 of EPSC differentiation, immunofluorescence staining of Brachyury was used to demonstrate that EPSCs were differentiated into mesoblastema (FIG. 2A). On Day 12 of EPSC differentiation, immunofluorescence staining was performed using myocardial precursor cell markers ISL1 and NKX2.5, demonstrating that the cells at this stage were myocardial precursor cells (FIG. 2B). On Day 16 of EPSC differentiation, co-staining with cardiomyocyte marker α-actinin and fibroblast marker Vimentin determined that the vast majority of the differentiated cells were cardiomyocytes (FIG. 2C); further, after the cells on Day 5 were digested and differentiated, the detection of mesoderm marker Brachyury by the flow cytometry found that the Brachyury positive rate of the cell population was 98.8% (FIG. 2D); after the cells on Day 12 were digested and differentiated, the detection of cardiomyocyte and myocardial precursor cell markers by the flow cytometry found that the ISL1 positive rate of the cell population was 93.5%, and the NKX2.5 positive rate thereof was 90% (FIG. 2E); and after the cells on Day 16 were digested, the percentage of the cardiomyocyte marker cTnT+ cell was up to 96.6% (FIG. 2F). By counting the cTnT positive rate of cardiomyocytes differentiated from eight batches of EPSCs, it was found that the cardiomyocyte differentiation efficiency of EPSCs was at least 80% each time, and the average differentiation efficiency was 88% (FIGS. 3A-B). These results showed that the differentiation efficiency of the differentiation system was high and stable.

### Example 4

After EPSCs and hiPSCs were digested, the cells were inoculated into 24-well plates at different densities of 1×10⁴ cells/cm², 2×10⁴ cells/cm², 4×10⁴ cells/cm², 6×10⁴ cells/cm², 8×10⁴ cells/cm². Each group included three replicates. EPSCs were resuspended in EPSC medium for one day and transformed and cultured in mTeSR™ 1 for two days. hiPSCs were resuspended and cultured in mTeSR™ 1 directly until Day 3. The percentage of the cell coverage area of EPSCs and hiPSCs per well in the total well area was measured by photographing and recorded as the initial differentiation confluence. Next, CHIR99021 (7.5 μM) induced the initial differentiation. On Day 16, videos were recorded to measure the percentages of the stem cell-differentiated myocardial beat regions of two groups in the total well area. According to the statistical results, when hiPSCs reached 70-85% cell confluence, the cardiomyocytes differentiation efficiency was the highest, and the beat region could reach 100%; when the differentiation density did not fall within the optimum density, the differentiation efficiency was significantly reduced. When EPSCs reached at least 80% initial differentiation confluence, the cardiomyocytes beat region could reach 100%, showing that the EPSCs had a wider differentiation potential and a better differentiation effect.

### Example 5

Comparison of Functions of EPSC-Differentiated Cardiomyocytes Versus Naïve Induced Pluripotent Stem Cell hiPSC-Differentiated Cardiomyocytes

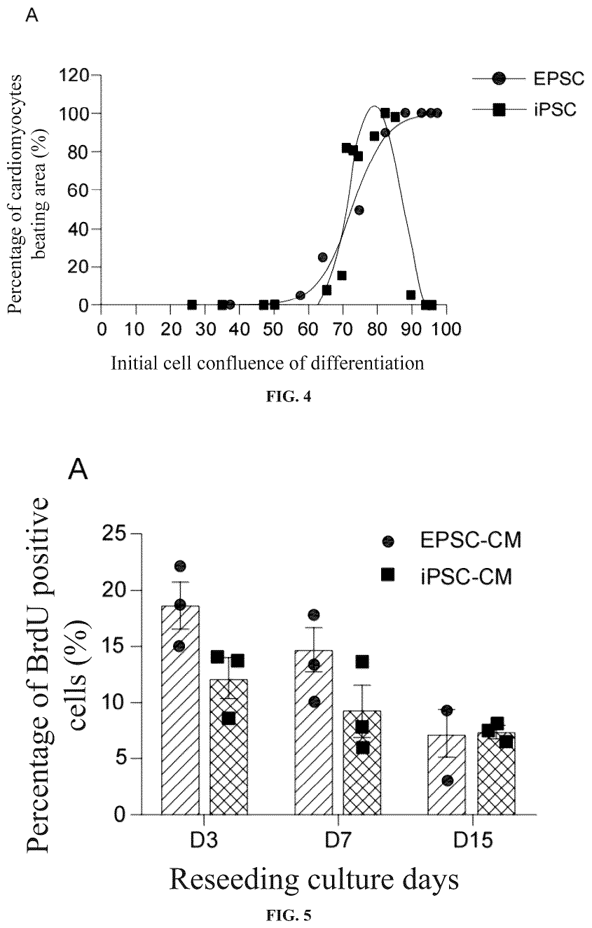

After the EPSCs induced cardiomyocyte differentiation to Day 16, a single cell was obtained by digesting with collagenase I and Trypsin-EDTA, re-seeded onto a coverslip or confocal dish. A 1:1 re-seeded culture medium was prepared from 1640+B27 and sugar-free DMEM+4 μM lactate. The cell was labeled with bromodeoxyuridine (BrdU) for 24 h on Days 3, 7, and 15. After cell immobilization on Days 4, 8, and 16 after re-seeding, α-actinin was stained immunofluorescent marked cardiomyocytes, and the proliferation percentage of cardiomyocytes was counted. The results showed that the proliferation rate of EPSC-CMs was slightly higher than that of hiPSC-CMs on Days 3 to 4 and 7 to 8 after reseeding, and the proliferation rate was consistent on Days 15 to 16 after the prolong culture (FIG. 5). After the cardiomyocytes differentiated under the same size plate well area and culture time were digested, the average number of EPSC-differentiated cardiomyocytes was (1-1.2)×10⁶ cells/cm², while the average number of hiPSC-differentiated cardiomyocytes was only (0.3-0.5)×10⁶ cells/cm².

### Example 6

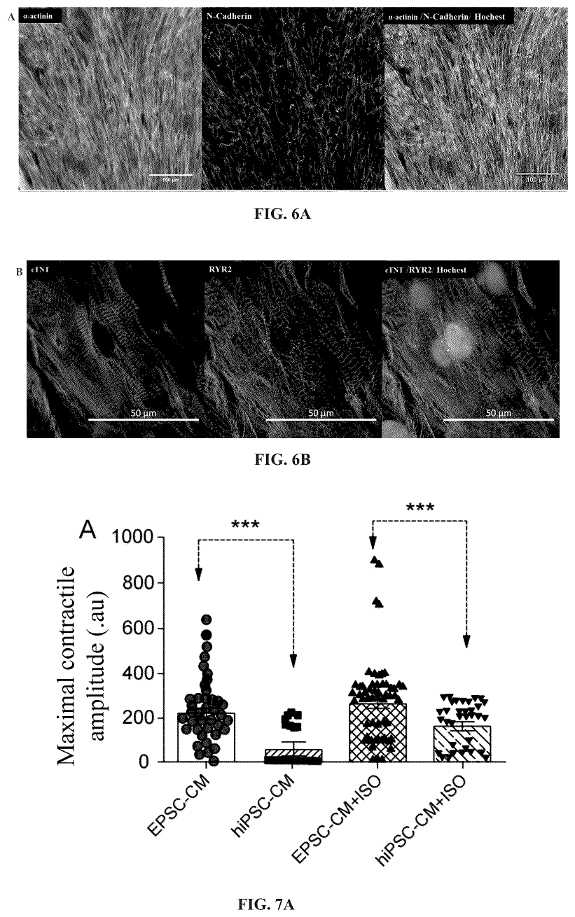

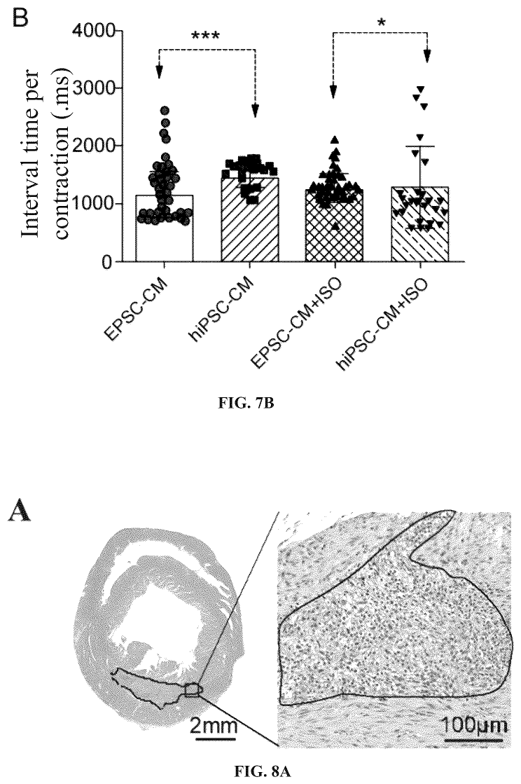

Due to individual differences in re-spread monolayer cells, their uniformity of function test is far less stable as that of tissues. In order to test the differences in function between EPSC- and hiPSC-differentiated cardiomyocytes, both groups were differentiated for 14 days and digested into single cells, followed by construction of strip-shaped engineering heart microtissues (EHT) self-prepared in the present laboratory. Each EHT strip was composed of 0.4×10 6 cells. Mixing with Matrigel, thrombin, and fibrinogen, beating EHT were generally visible to the naked eye on Days 3 to 5 after construction. After EHT culture was carried out until Day 14, immunofluorescence staining with cardiomyocyte markers α-actinin and cTnI and myocardial maturation markers N-cadherin and RYR2 found that EPSC-differentiated cardiomyocytes were arranged regularly, N-cadherin at the cell junction was partly distributed to two polar, and the RYR2 staining presented a definite structure (FIGS. 6A-B). Meanwhile, a video of microtissue contraction was recorded, and the contractility of either group was analyzed by Image J plug-in Myociter v1.3. As a result, the functional contractility of EPSC-CM was significantly higher than that of hiPSC-CM (FIGS. 7A-B), specifically showing that the maximal contractile amplitude was significantly higher than that of hiPSC-CMs (FIG. 7A), while the contraction frequency was higher (FIG. 7B); and the difference was more significant under the stimulation of the ISO stimulating. To sum up, it was believed that the maturity of EPSC-differentiated cardiomyocytes was higher than that of hiPSC-CMs.

### Example 7

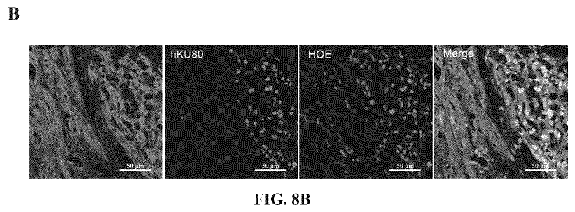

A nude rat aged 8 weeks was anesthetized, and thoracotomy was performed. EPSC-CMs were injected into the muscular layer of the myocardium in the left ventricular anterior wall in a multipoint manner. The chest was closed layer by layer so that the rat came round. The heart was eviscerated for pathological examination after the rat was fed for four weeks. H&E staining showed that a plurality of EPSC-CMs resided and survived in myocardial tissue (FIG. 8A). Further immunofluorescence staining demonstrated that the resident cells were human cardiomyocytes and the sarcomere structure was complete (FIG. 8B). The above results showed that EPSC-CMs could effectively survive in the myocardium after cell transplantation, and can be used in cell therapy for cardiomyopathy.

The description of above examples is merely provided to help understand the method of the present disclosure and a core idea thereof. It should be noted that several improvements and modifications may be made by a person of ordinary skill in the art without departing from the principle of the present disclosure, and these improvements and modifications should further fall within the protection scope claimed by the present disclosure. A plurality of modifications to these examples are apparent to those skilled in the art, and the general principle defined herein may be practiced in other examples without departing from the spirit or scope of the present disclosure. Accordingly, the present disclosure will not be limited to these examples shown herein, but is to fall within the widest scope consistent with the principles and novel features disclosed herein.

What is claimed is:

1. A method for preparing a cardiomyocyte, wherein the cardiomyocyte is prepared from an extended pluripotent stem cell (EPSC).

2. The method for preparing a cardiomyocyte according to claim 1, wherein the method comprises induced differentiation of the EPSC into the cardiomyocyte.

3. The method for preparing a cardiomyocyte according to claim 2, wherein the induced differentiation is implemented by inducing the EPSC to differentiate into the cardiomyocyte through small molecule-based phased regulation of a Wnt signaling pathway.

4. A method for induced differentiation of an EPSC into a cardiomyocyte, wherein the method is implemented by inducing the EPSC to differentiate into the cardiomyocyte through small molecule-based phased regulation of a Wnt signaling pathway.

5. The method according to claim 4, wherein the method is implemented by inducing the EPSC to differentiate into the cardiomyocyte through the small molecule-based phased regulation of the Wnt signaling pathway, and before the small molecule-based phased regulation, the method further comprises rapid transition and transformation of the EPSC via mTeSR™ 1 and/or KnockOut™ DMEM/F-12+B27+fibroblast growth factor 2 (FGF2)+transforming growth factor-β (TGFβ).

6. The method according to claim 5, wherein the B27 comprises insulin; and culture time for the rapid transition and transformation is 1-4 days.

7. The method according to claim 5, wherein on condition that the EPSC reaches at least 50% confluence, the induced differentiation is initiated to form the cardiomyocyte.

8. The method according to claim 7, wherein the EPSC reaches at least 85% confluence.

9. The method according to claim 7, wherein the EPSC reaches at least 95% confluence.

10. The method according to claim 4, wherein the small molecule-based phased regulation specifically comprises steps of: adding a small molecule CHIR99021 for culturing; and adding Wnt signaling pathway-inhibiting small molecules IWR and IWP2 for culturing.

11. The method according to claim 10, wherein a concentration of the CHIR99021 is 5-10 μM.

12. The method according to claim 11, wherein the concentration of the CHIR99021 is 7.5 μM.

13. The method according to claim 10, wherein a concentration of the IWR is 2.5-7.5 μM.

14. The method according to claim 13, wherein the concentration of the IWR is 5 μM.

15. The method according to claim 10, wherein a concentration of the IWP2 is 1-5 μM; and
preferably, the concentration of the IWP2 is 2.5 μM.

16. The method according to claim 10, wherein a cell culture medium used during the small molecule-based phased regulation comprises 1640+B27, and the B27 is insulin-free.

17. The method according to claim 10, wherein culture time during the small molecule-based phased regulation is 3-7 days, and preferably 4-5 days.

18. A cardiomyocyte obtained by the method according to claim 3.

19. The cardiomyocyte according to claim 18, wherein the method is implemented by inducing the EPSC to differentiate into the cardiomyocyte through small molecule-based phased regulation of a Wnt signaling pathway.

20. The cardiomyocyte according to claim 19, wherein the method is implemented by inducing the EPSC to differentiate into the cardiomyocyte through the small molecule-based phased regulation of the Wnt signaling pathway, and before the small molecule-based phased regulation, the method further comprises rapid transition and transformation of the EPSC via mTeSR™ 1 and/or KnockOut™ DMEM/F-12+B27+fibroblast growth factor 2 (FGF2)+transforming growth factor-β (TGFβ).
